(19) United States
(12) Patent Application Publication
(10) Pub. No.: US 2016/0074333 A1
(43) Pub. Date: Mar. 17, 2016

(54) Pharmaceutical compositions of tamsulosin or salts thereof

(71) Applicant: WOCKHARDT LIMITED, Aurangabad O, (IN)

(72) Inventors: Rahul Sudhakar DABRE, Nagpur 440015, (IN); Girish Kumar JAIN, Delhi 4, (IN); Inderjeetsingh HUDA, Aurangabad 5, (IN); Vijay SUGGALA, Khammam 507001, (IN); Yatendra KUMAR, Aurangabad 3, (IN)

(21) Appl. No.: 14/767,625

(22) PCT Filed: Jun. 14, 2014
(86) PCT No.: PCT/IB2014/062232

§ 371 Date: Aug. 13, 2015

(30) Foreign Application Priority Data
Jun. 21, 2013 (IN) 2100/MUM/2013
Jun. 21, 2013 (IN) 2101/MUM/2013

(51) Int. Cl. A61K 9/50; A61K 9/48; A61K 9/16; A61K 31/18; A61K 31/473

(52) U.S. Cl. CPC A61K 9/5026; A61K 31/18; A61K 31/473; A61K 9/5073; A61K 9/1652; A61K 9/1635; A61K 9/4808; A61K 9/4866

(57) ABSTRACT

The present invention relates to pharmaceutical compositions of tamsulosin or salts thereof. In particular, the invention relates to pharmaceutical compositions comprising a core of tamsulosin or salts, hydrates thereof and at least one specialized coating over the core. Such compositions of tamsulosin may exhibit desired release kinetics with excellent storage stability and particularly, levels of degradants in the formulation during storage can be effectively controlled. The invention also includes a process of preparing such compositions and additionally combination with other pharmaceutical ingredient.

### FIELD OF THE INVENTION

The present invention relates to pharmaceutical compositions of tamsulosin or salts thereof. In particular, the invention relates to pharmaceutical compositions comprising a core of tamsulosin or salts, hydrates thereof and at least one specialized coating over the core. Such compositions of tamsulosin may exhibit desired release kinetics with excellent storage stability and particularly, levels of degradants in the formulation during storage can be effectively controlled. The invention also includes a process of preparing such compositions and additionally combination with other pharmaceutical ingredient.

### BACKGROUND OF THE INVENTION

Tamsulosin is a selective α₁ receptor antagonist that has preferential selectivity for the α₁A receptor in the prostate versus the α₂B receptor in the blood vessels.

When α₁A receptors in the bladder neck and the prostate are blocked, this causes a relaxation in smooth muscle and therefore less resistance to urinary flow. Due to this the pain associated with BPH can be reduced.

Tamsulosin hydrochloride is (-)-(R)-5-[2-[[2-(o-Ethoxyphenoxy) ethyl]amino] propyl]-2-methoxybenzene sulfonamide, monohydrochloride. Tamsulosin hydrochloride is a white crystalline powder that melts with decomposition at approximately 230° C. It is sparingly soluble in water and methanol, slightly soluble in glacial acetic acid and ethanol, and practically insoluble in ether. The empirical formula of tamsulosin hydrochloride is C₂₀H₂₈N₂O₅S. HCl. The molecular weight of tamsulosin hydrochloride is 444.98. Its structural formula is:

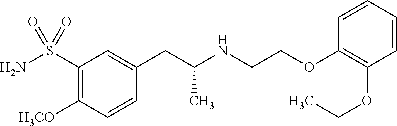 [SMILES: CCOc1ccccc1OCCN[C@H](C)Cc1ccc(OC)c(S(N)(=O)=O)c1]

Tamsulosin product is commercially available under the trade name Flomax® and Omnic® in the US and in Europe respectively.

In a medical treatment, it is advantageous to administer tamsulosin together with another active substance. Such another substance might be of the same or different therapeutic class and may act in a synergistic way with tamsulosin. The administration of two drugs in a single dosage form is most preferred dosage form due to ease of administration. Combination of tamsulosin with dutasteride available commercially in the US under the brand name Jalyn™. Jalyn™ (dutasteride and tamsulosin hydrochloride) Capsules are indicated for the treatment of symptomatic benign prostatic hyperplasia, or benign prostatic hypertrophy (BPH) in men with an enlarged prostate.

U.S. Pat. No. 4,772,475 discloses pellets of tamsulosin prepared and tested for release characteristics. The test results showed that in one hour the release ranged from 16.2 to 60.4% of the active compound using simulated gastric fluid as dissolution media. Further, tablets made from produced pellets, showed 50.3 and 57.6% release, respectively, were also tested on human volunteers in comparison with conventional tablets and concentration of the active substance in blood plasma was measured. Peak plasma levels were reached 3 hours after ingestion (in comparison with 2 h at conventional tablets), the total amount of tamsulosin in plasma being about 75% of that of the conventional tablet. However such release is not sufficient for an extended-release dosage form.

U.S. Pat. No. 7,018,658 discloses pharmaceutical pellets comprising tamsulosin. The pellets have a pellet core comprises tamsulosin hydrochloride, microcrystalline cellulose, polymer and water and an outer enteric coat.

PCT Publication No. WO 2006/055659 discloses fixed dose pharmaceutical compositions of dutasteride and tamsulosin. Dutasteride is a dual, azasteroid, 5-α-reductase inhibitor approved currently for the treatment of patients with moderate to severe symptoms of BPH. Dutasteride is commercially available from GlaxoSmithkline and is marketed in US under the brand name Avodart®.

U.S. Patent Application Publication No. 2011/0244033 discloses tamsulosin pellets for fixed dose combination containing physically separated tamsulosin dose and testosterone 5α-reductase inhibitor for oral administration.

Although various compositions have been made earlier for improving the release rate of the tamsulosin, the inventors have further found that when tamsulosin is combined with other active pharmaceutical ingredient such as dutasteride for fixed dose combination formulation, the resulting formulation may have compromised stability over the storage period. Thus, there still exists an enduring need for alternative and improved pharmaceutical composition of tamsulosin, or its fixed dose combination formulation with other active pharmaceutical ingredient, which can exhibit desired release profile, and alongside, posses excellent storage stability.

The inventors of the present invention have surprisingly found that it is possible to formulate a pharmaceutical composition of tamsulosin, which can address aforesaid objectives. Particularly, the inventors have found that by using specialized coating over the tamsulosin cores, the resulting formulation exhibits desired release profile and excellent storage stability, peculiarly when it is combined with other active pharmaceutical ingredients.

### SUMMARY OF THE INVENTION

In one general aspect, there is provided a pharmaceutical composition comprising:

(a) at least one core comprising tamsulosin, or salts, hydrates thereof;

(b) at least one enteric coating layer coated over the core comprising one or more pharmaceutically acceptable acid resistant polymers,

wherein the mass of said enteric coating layer, calculated on the uncoated core basis is more than 15%.

In another general aspect, there is provided a pharmaceutical composition comprising:

(a) at least one tablet comprising tamsulosin, or salts, hydrates thereof;

(b) at least one enteric coating layer coated over the tablet comprising one or more pharmaceutically acceptable acid resistant polymers,

wherein the mass of said enteric coating layer, calculated on the uncoated tablet basis is more than 15%.

In another general aspect, there is provided a capsule composition comprising:

(a) at least one core comprising tamsulosin, or salts, hydrates thereof;

(b) at least one enteric coating layer coated over the core comprising one or more pharmaceutically acceptable acid resistant polymers,

wherein the mass of said enteric coating layer, calculated on the uncoated core basis is more than 15%.

In another general aspect, there is provided a pharmaceutical composition comprising:

(a) at least one core comprising tamsulosin, or salts, hydrates thereof;

(b) at least one enteric coating layer coated over the core comprising one or more pharmaceutically acceptable acid resistant polymers; and

(c) at least one moisture barrier coating layer disposed over the enteric coating layer comprising one or more pharmaceutical polymers,

wherein the mass of said enteric coating layer, calculated on the uncoated core basis is more than 15%.

In another general aspect, there is provided a pharmaceutical composition comprising:

(a) at least one core comprising tamsulosin, or salts, hydrates thereof;

(b) at least one enteric coating layer coated over the core comprising one or more pharmaceutically acceptable acid resistant polymers; and

(c) at least one moisture barrier coating layer disposed over the enteric coating layer comprising one or more pharmaceutical polymers,

wherein the mass of said enteric coating layer, calculated on the uncoated core basis is more than 15% and characterized in that said composition exhibits a dissolution release profile in simulated gastric fluid (SGF) using Ph. Eur. Basket method at 100 rpm such that not more than about 10% of tamsulosin is released in first 2 hours.

In another general aspect, there is provided a fixed dose combination composition comprising: a first composition which comprises of—

(a) at least one core comprising tamsulosin, or salts, hydrates thereof;

(b) at least one enteric coating layer coated over the core comprising one or more pharmaceutically acceptable acid resistant polymers; and

(c) at least one moisture barrier coating layer disposed over the enteric coating layer comprising one or more pharmaceutical polymers,

and a second composition which comprises of dutasteride or salts, hydrates thereof; characterized in that the mass of said enteric coating layer, calculated on the uncoated core basis is more than 15% and the second composition is physically separated from the first composition.

In another general aspect, there is provided a capsule comprising: a first composition which comprises of—

(a) at least one core comprising tamsulosin, or salts, hydrates thereof;

(b) at least one enteric coating layer coated over the core comprising one or more pharmaceutically acceptable acid resistant polymers;

(c) at least one moisture barrier coating layer disposed over the enteric coating layer comprising one or more pharmaceutical polymers;

and a second composition which comprises of dutasteride or salts, hydrates thereof, characterized in that the mass of said enteric coating layer, calculated on the uncoated core basis is more than 15%, the second composition is physically separated from the first composition, and said capsule exhibits a dissolution release profile in simulated gastric fluid (SGF) using Ph. Eur. Basket method at 100 rpm such that not more than about 10% of tamsulosin is released in first 2 hours.

In another general aspect there is provided a process for preparing a pharmaceutical composition of tamsulosin or salts, hydrates thereof, which process comprising steps of:

(a) providing at least one core; comprising tamsulosin hydrochloride, or salts hydrates thereof;

(b) providing at least one enteric coating layer over the core comprising one or more pharmaceutically acceptable acid resistant polymers; and

(c) providing at least one coating layer over the enteric coating layer providing moisture barrier layer comprising one or more pharmaceutical polymers,

wherein the mass of said enteric coat, calculated on the uncoated core basis is more than 15%.

In another general aspect there is provided a process for preparing of a pharmaceutical composition of tamsulosin or salts, hydrates thereof processes comprising steps of:

(a) providing at least one core; comprising tamsulosin hydrochloride, or salts hydrates thereof;

(b) providing at least one enteric coating layer coated over the core comprising one or more pharmaceutically acceptable acid resistant polymers, wherein the mass of said enteric coating layer, calculated on the uncoated core basis is more than 15%;

(c) providing at least one coating layer disposed over the enteric coating layer providing moisture barrier layer comprising one or more pharmaceutical polymers to form a first composition;

(d) providing a second composition comprising dutasteride or salts, hydrates thereof; and

(e) formulating the first and second compositions in the unit dosage form.

In another general aspect there is provided a method of treating the symptoms of benign prostatic hyperplasia, which comprises administering a pharmaceutical composition of tamsulosin or salts, hydrates thereof to a patient in need thereof, which composition comprises of:

(a) at least one core comprising tamsulosin, or salts, hydrates thereof;

(b) at least one enteric coating layer coated over the core comprising one or more pharmaceutically acceptable acid resistant polymers;

(c) optionally, at least one moisture barrier coating layer disposed over the enteric coat layer comprising one or more pharmaceutical polymers,

wherein the mass of said enteric coating layer, calculated on the uncoated core basis is more than 15%.

In another general aspect there is provided a method of treating the symptoms of benign prostatic hyperplasia, which comprises administering an effective amount of the pharmaceutical composition to a patient in need thereof comprising:

(a) at least one core comprising tamsulosin, or salts, hydrates thereof;

(b) at least one enteric coating layer coated over the core comprising one or more pharmaceutically acceptable acid resistant polymers;

(c) at least one moisture barrier coating layer disposed over the enteric coating layer comprising one or more pharmaceutical polymers,

and a second composition which comprises of dutasteride or salts, hydrates thereof;
characterized in that the mass of said enteric coating layer, calculated on the uncoated core basis is more than 15% and the second composition is physically separated from the first composition.

### DETAILED DESCRIPTION OF THE INVENTION

The term “tamsulosin” used throughout the specification refers to not only tamsulosin but also various pharmaceutically acceptable salts and pharmaceutically acceptable solvates thereof or can be mixture thereof. The tamsulosin is preferably tamsulosin hydrochloride.

The term “core” used throughout the specification refers to a “core”, “pellet” “tablet core”, “tablet core composition” or “core composition” “bead” or “plurality of beads”.

Enteric coating is applied onto the unit dosage forms in which a tablet or core is coated with a material which prevent or minimize release in the stomach but allow release in the small intestine. This type of formulation either protects the stomach from a potentially irritating drug or protects the drug from partial degradation in the acidic environment of the stomach or drug which is absorbed from the small intestine.

The pharmaceutical composition of the present invention comprises tamsulosin, salts, hydrates thereof. The composition comprises one or more core and at least one enteric coated layers on the core, and the first enteric coat layer comprises pharmaceutically acceptable acid resistant polymer. The mass of said enteric coat, calculated on the uncoated core basis is more than 15%.

In an embodiment, the composition exhibits a dissolution release profile in simulated gastric fluid (SGF) using Ph. Eur. Basket method at 100 rpm such that not more than about 10% of tamsulosin is released in first 2 hours.

The core may be formed of one or more pharmaceutical excipients selected from, but not limited to one or more of bulking agents or fillers, binders, disintegrants, and lubricants.

The bulking agents or fillers may be present in the core in an amount within the range from about 1 to about 95% w/w and preferably from about 10 to about 85% w/w of the composition. Examples of bulking agents or fillers suitable for use herein include, but are not limited to, cellulose derivatives such as microcrystalline cellulose or wood cellulose, lactose, sucrose, starch, pregelatinized starch, dextrose, mannitol, fructose, xylitol, sorbitol, corn starch, modified corn starch, inorganic salts such as calcium carbonate, calcium phosphate, dicalcium phosphate, calcium sulfate, dextrin/dextrates, maltodextrin, compressible sugars, and other known bulking agents or fillers, and/or mixtures thereof, preferably microcrystalline cellulose.

The binder may be present in the core in an amount within the range from about 0 to about 20% w/w, preferably from about 1 to about 10% w/w of the composition. Examples of binders suitable for use herein include, but are not limited to, hydroxypropyl cellulose, corn starch, pregelatinized starch, modified corn starch, polyvinyl pyrrolidone (PVP) (molecular weight ranging from about 5,000 to about 1,000,000, preferably about 40,000), hydroxypropyl methylcellulose (HPMC), lactose, gum acacia, ethyl cellulose, cellulose acetate, as well as a wax binder such as carnauba wax, paraffin, spermaceti, polyethylenes or microcrystalline wax, as well as other conventional binding agent and/or mixtures thereof, preferably hydroxypropyl cellulose.

The disintegrant may be present in the core in an amount within the range from about 0 to about 20% w/w, preferably from about 0.25 to about 10% w/w of the composition. Examples of disintegrants suitable for use herein include, but are not limited to, croscarmellose sodium, crospovidone, starch, potato starch, pregelatinized starch, corn starch, sodium starch glycolate, microcrystalline cellulose, low substituted hydroxypropyl cellulose or other known disintegrant, preferably croscarmellose sodium.

The lubricant may be present in the core in an amount within the range from about 0.1 to about 5% w/w, preferably from about 0.2 to about 2% w/w of the composition. Examples of tableting lubricants suitable for use herein include, but are not limited to, magnesium stearate, zinc stearate, calcium stearate, talc, carnauba wax, stearic acid, palmitic acid, sodium stearyl fumarate or hydrogenated vegetable oils and fats, or mixtures thereof, preferably magnesium stearate.

In another embodiment, the bulking agents are microcrystalline cellulose, the disintegrant is pregelatinized starch, and the lubricant/glidant is colloidal silica.

In an embodiment, the core preferably contain at least one bulking agent or filler; optionally at least one binder; optionally at least one disintegrant; and preferably but optionally at least one lubricant. The bulking agent or filler may present in an amount within the range from about 1 to about 95% w/w, preferably from about 10 to about 85% w/w of the composition. The binder may present in an amount within the range from about 0 to about 20% w/w, preferably from about 1 to about 10% w/w of the composition. The disintegrant may present in an amount within the range from about 0 to about 20% w/w, and preferably from about 0.25 to about 10% w/w of the composition. The lubricant may present in an amount within the range from about 0 to about 5% w/w, preferably from about 0.2 to about 2% w/w of the composition.

The cores present in the composition of the invention can be prepared by a variety of processes and order of addition of excipients. The utility of these formulations is not limited to a specific dosage form or manufacturing process. The cores may be manufactured by wet granulation, dry granulation, direct blending or any other pharmaceutically acceptable process.

In an embodiment, the process of preparing the cores includes the steps of blending one or more excipients such as bulking agent, optionally binder and optionally disintegrant. A lubricant will be preferably added to the blend to facilitate core formation.

In another embodiment, the process of preparing the cores includes the steps of coating the dispersion comprising aspirin and one or more pharmaceutical excipients over inert beads of sugar or microcrystalline cellulose.

Examples of polymers suitable for seal coating layer include, but not limited to acrylate copolymers, hydroxypropyl methylcellulose, ethyl cellulose, methacrylic polymers or hydroxypropyl cellulose, preferably hydroxypropyl methylcellulose. The coating layer may also optionally include a plasticizer such as triacetin, diethyl phthalate, tributyl sebacate or polyethylene glycol (PEG), preferably PEG; and an anti-adherent or glidant such as talc, fumed silica or magnesium stearate, opacifying agent such as titanium dioxide. The coating layer may also include iron oxide based colorants.

Examples of suitable coating solvents includes, but not limited to water, ethanol, methanol, and isopropyl alcohol, with water being preferred.

The enteric coatings work by presenting a surface that is not soluble in acidic aqueous medium while soluble in neutral or basic aqueous medium. Enteric coating agent is an excipient that allows releasing the active substance from the composition only under certain environmental condition and or by a certain release rate.

Enteric coating is applied onto the unit dosage forms as solutions in organic or aqueous solvents. The solvents commonly employed as vehicles are water, methylene chloride, ethanol, methanol, isopropyl alcohol, acetone, ethyl acetate and combinations thereof. The choice of the solvent is based primarily on the solubility of the polymer, ease of evaporation, and viscosity of the solution.

Acid resistant polymers suitable for enteric coating layer may be selected from, but not limited to acrylic acid polymers, phthalate polymers, copolymers or mixtures thereof. An acrylic polymer such as polyacrylates or polymethacrylates is the preferred polymer for enteric coating layer applied over the cores.

Polyacrylates, the polymethacrylates and the copolymers of acrylic and methacrylic acid are commercially available under the name Eudragit® and are particularly suitable. Examples of Eudragit® products include Eudragit L30D, Eudragit® L30 and Eudragit® D-55 etc. Other suitable enteric polymers include, for example, cellulose derivates such as, cellulose acetate phthalate, hydroxypropyl methylcellulose phthalate, polyvinyl acetate phthalate, etc.

Furthermore enteric coating polymers may be combined together in order to induce both time dependent and pH dependent control of the release of tamsulosin. The amount of enteric coating calculated on the uncoated core basis is more than 15%, preferably more than 18%, more preferably more than 20%.

The enteric coating layer of the composition comprises one or more pharmaceutical excipients, pharmaceutically acceptable acid resistant acrylic polymers and optionally one or more polymers.

The amount of acid resistant enteric polymer in the coating layer is more than 50% by weight of the coating, In an embodiment more than 70% by weight of coating, and in a further embodiment more than 80% by weight relative to the total weight of the coating layer.

The amount of acid resistant polymer is preferably within the range of 20-85 mass %, more preferably 30 to 75%, and typically 50 to 75%, calculated on a dry basis of the coating layer.

The coating formulation for enteric coating contains at least one polymer and a coating solvent, which preferably is water, which is used for processing and removed by drying. Suitable polymer for first coating layer may be selected from, but not limited to pharmaceutically acceptable acid resistant acrylic polymers, water-soluble polymer, water-insoluble polymer, or mixtures thereof. Particularly, water-soluble polymers are preferred.

The enteric coating layer which is pharmaceutically acceptable acid resistant acrylic polymers formed by polymer layer in an amount within the range from about 15 to about 90%, preferably from about 20 to about 90% w/w, more preferably 20 to 80% of the enteric coating layer, optionally plasticizer in an amount within the range from about 1 to about 30%, preferably from about 5 to about 20% w/w of the enteric coating layer, and anti-adherent or glidant in an amount within the range for about 5 to about 30%, preferably from about 10 to about 15% w/w of the enteric coating layer.

The enteric coating layer may be present in an amount within the range from about 15 to about 50%, preferably from about 15 to about 25% w/w of the composition.

In addition to enteric coating layer there is additional second layer of coating disposed over the enteric coating which provides moisture barrier for the core.

The inventors of the present invention have surprisingly found that by applying the moisture barrier layer, the resulting composition may exhibit excellent storage stability, peculiarly when it is combined with other active pharmaceutical ingredients such as dutasteride.

The stability study composition may exhibit chemical and/or physical stability such that the composition retains at least 90% w/w of tamsulosin when stored at 40° C. and 75% relative humidity after storing for at least 3 months.

The moisture barrier layer comprising one or more pharmaceutical polymers from various classes such as polyvinyl alcohol, hydroxypropyl methyl cellulose such as OPADRY® AMB (Aqueous Moisture Barrier); which is a preferred barrier material and is commercially available from colorcon; one or more pharmaceutical excipients and optionally, one or more polymers. Suitable coating solvent is employed to facilitate the coating, which preferably is water, which is used for processing and may be removed by drying.

The moisture barrier layer may be present in an amount within the range from about 1 to about 25%, preferably from about 1 to about 15% w/w of the composition.

The coating layers of the composition may include one or more pharmaceutical excipients comprising bulking agents or diluents, binders, plasticizers, lubricants, colorants, pH adjusting agents, or mixtures thereof.

The composition of the present invention may be formulated in suitable a dosage form including, but not limited to, a tablet, caplet, mini-tablet, pellets, granules, capsule filled with mini-tablets or pellets or combinations thereof.

In an embodiment, the pharmaceutical composition comprising plurality of pellets, which pellets comprises of about 0.05 to about 5.0% w/w of tamsulosin hydrochloride, about 50 to about 85% w/w of microcrystalline cellulose, about 10 to about 25% w/w of the acrylic polymer, about 5 to about 20% w/w polyvinyl alcohol based polymer, about 2 to about 10% w/w of water, and about 0% to about 25% w/w of other pharmaceutical excipients, calculated on the dry pellet basis.

The pharmaceutical composition optionally contain at least second active ingredient from testosterone 5α-reductase inhibitor class which is physically separated from coated core; and said testosterone 5α-reductase inhibitor is dutasteride. Such fixed dose combination composition may be used in a medical treatment e.g. in a treatment of benign prostatic hyperplasia.

In an embodiment, the fixed dose combination composition comprises a first composition which comprises of—

(a) at least one core comprising tamsulosin, or salts, hydrates thereof;

(b) at least one enteric coating layer coated over the core comprising one or more pharmaceutically acceptable acid resistant polymers; and

(c) at least one moisture barrier coating layer disposed over the enteric coating layer comprising one or more pharmaceutical polymers,

and a second composition which comprises of dutasteride or salts, hydrates thereof.

Said dutasteride composition is formulated, preferably, in a soft gel composition and tamsulosin is formulated in the form of coated cores or pellets and then incorporated in capsule.

In capsule composition an outer capsule surrounding the inner capsule and forming a space between the inner and outer capsule and the space between the inner and outer capsule is filled with coated cores comprising tamsulosin, salts or hydrates thereof and second active ingredient is present in inner capsule in physically separated form.

In an embodiment, the mass of all the coating layers over the uncoated core is within the range of about 15 to 25% w/w calculated on the dry uncoated core basis.

Coatings in the composition of the invention may be achieved by methods known to one skilled in the art such as by using fluidized bed equipment, perforated pans, a regular pharmaceutical pan, compression coating, and continuous or short spray methods. For example, a plasticized dispersion of coating polymer may be applied onto the tablet core comprising the therapeutic active agent by spraying using any suitable spray equipment known in the art. In one embodiment the solid unit dosage forms are coated by continuous spray methods.

In an embodiment, the method of preparing the cores employed in the composition of the invention which includes the steps of blending the one or more excipients such as bulking agent, optionally binder and optionally disintegrant. A lubricant will be preferably added to the blend to facilitate the core formation.

The invention further provides a method of treating the symptoms of benign prostatic hyperplasia, which comprises administering an effective amount of the pharmaceutical composition as substantially described herein throughout the specification to a patient in need thereof.

The present invention is further illustrated by the following examples which are provided merely to be exemplary of the invention and do not limit the scope of the invention. Certain modifications and equivalents will be apparent to those skilled in the art and are intended to be included within the scope of the present invention.

### Example 1

### Tamsulosin Pellets

TABLE 1

| Sr. No. | Ingredients | Qty/Tab |
|---|---|---|
| Core pellets | | |
| 1 | Tamsulosin Hydrochloride | 0.40 |
| 2 | Microcrystalline Cellulose | 92.60 |
| 3 | Purified water | q.s. |
| Total | | 93.00 |
| Dough preparation | | |
| 4 | Microcrystalline Cellulose | 24.00 |
| 5 | Magnesium stearate | 3.00 |
| 6 | Eudragit L 30 D-55 | 30.00 |
| 7 | Purified water | |
| Weight of core pellets | | 150.00 |
| Extended release coating | | |
| 8 | Eudragit L 30 D-55 | 8.10 |
| 9 | Triethyl citrate | 0.90 |
| 10 | Purified water | |
| Weight of coated pellets | | 159.00 |
| 11 | Opadry white AMB | 19.08 |
| Lubrication | | |
| 12 | Talc | 0.42 |

| | |
|---|---|
| Total | 178.50 |

Process: Core pellets were prepared by granulating microcrystalline cellulose using purified water containing tamsulosin hydrochloride in rapid mixer granulator (RMG). The blend was dried in fluidized bed dryer (FBD) and granulated with Eudragit® L30 D55 in RMG. The wet mass then passed through 1.0 mm bore size and spheronizer to form spheres. The formed spheres were dried in dryer.

The first coating suspension prepared by blending Eudragit L30 D55 with triethyl citrate and purified water then coated over the core pellets and desired weight gain was achieved.

Opadry white AMB coating solution was then applied over the coated pellets followed by drying of the film coat.

### Example 2

### Tamsulosin Pellets

TABLE 2

| Sr. No. | Ingredients | Qty/Tab |
|---|---|---|
| Core pellets | | |
| 1 | Tamsulosin Hydrochloride | 0.40 |
| 2 | Microcrystalline Cellulose | 92.60 |
| 3 | Purified water | q.s. |
| Total | | 93.00 |
| Dough preparation | | |
| 4 | Microcrystalline Cellulose | 24.00 |
| 5 | Magnesium stearate | 3.00 |
| 6 | Eudragit L 30 D-55 | 30.00 |
| 7 | Purified water | q.s. |
| Weight of core pellets | | 150.00 |
| Extended release coating | | |
| 8 | Eudragit L 30 D-55 | 13.50 |
| 9 | Triethyl citrate | 1.50 |
| 10 | Purified water | q.s. |
| Weight of coated pellets | | 165.00 |
| 11 | Opadry white AMB | 13.20 |
| Lubrication | | |
| 12 | Talc | 0.30 |

| | |
|---|---|
| Total | 178.50 |

Process: Tamsulosin pellets were by the process prepared as per Example 1.

### Example 3

### Capsule Containing Tamsulosin Pellets and Dutasteride Soft Gelatin Capsule

TABLE 3

| | | |
|---|---|---|
| | | Composition |
| Sr. No | Ingredients | (mg/capsule) |

Fill composition

| | | |
|---|---|---|
| 1 | Dutasteride | 0.500 |
| 2 | Mono & Di-glycerides | 109.665 |
| | (Imwitor 742) | |
| 3 | Medium chain triglycerides | 89.800 |
| | (Miglyol 812) | |
| 4 | Butylated hydroxytoluene | 0.035 |

| | |
|---|---|
| Total weight of fill solution | 200.00 |

Composition of shell

| | | |
|---|---|---|
| 5 | Gelatin | 116.935 |
| 6 | Glycerin | 53.93 |
| 7 | Titanium dioxide | 1.90 |
| 8 | Ferric oxide yellow | 0.235 |

| | |
|---|---|
| Final weight of Soft Gelatin Capsule | 373.00 |

Process: Dutasteride, mono & di-glycerides, medium chain triglycerides and butylated hydroxytoluene were mixed together and filled in soft gelatin capsule using a capsule filling machine. Tamsulosin pellets were prepared as per Example 1 or 2. Finally, tamsulosin pellets and a dutasteride soft gelatin capsule were the filled in hydroxypropyl methylcellulose capsules using a capsule filling machine.

### Dissolution Study:

(1) Tamsulosin part: Capsules containing tamsulosin pellets prepared according to Example 1 or 2 were subjected to dissolution first in acid stage for initial 2 hrs in 750 ml, 0.1 N HCl media using USP type II dissolution apparatus at 50 rpm speed of the basket, followed by buffer stage in 250 ml of pH 6.8 phosphate buffer using USP type II dissolution apparatus at 50 rpm speed of the basket. Table 2 summarizes the results of the dissolution study.

TABLE 4

| Time | % Tamsulosin Release | | |
|---|---|---|---|
| (Hours) | Initial | 1 M, 40° C./75% RH | 2 M, 40° C./75% RH |
| 0.5 | 33 | 39 | 32 |
| 1 | 49 | 56 | 47 |
| 2 | 70 | 78 | 65 |
| 3 | 84 | 91 | 76 |
| 4 | 92 | 98 | 84 |
| 5 | 98 | 100 | 89 |
| 7 | 98 | 102 | 91 |
| 10 | 98 | 102 | 92 |
| Assay | 99 | 99.5 | — |

(1) Dutasteride part: Capsules containing dutasteride soft gelatin capsule prepared according to Example 2 were subjected to dissolution in 900 ml media containing 0.1N HCl, 0.16% pepsin and 1% w/v of cetyltrimethyl ammonium bromide using USP type II dissolution apparatus at 75 rpm speed of the basket. Table 2 summarizes the results of the dissolution study.

TABLE 5

| Time | % Dutasteride Release | | |
|---|---|---|---|
| (Minutes) | Initial | 1 M, 40° C./75% RH | 2 M, 40° C./75% RH |
| 10 | 0 | ND | 0 |
| 15 | 16 | ND | 1 |
| 20 | 28 | ND | 7 |
| 30 | 70 | ND | 36 |
| 45 | 89 | ND | 82 |
| 60 | 89 | ND | 94 |

ND: Not determined

### Stability Study:

Capsules prepared according to Example 2 were subjected to stability study by placing HDPE bottles filled with the capsules over a period of 2 months at 40° and 70% relative humidity. Table 5 and 6 below summarizes results of the stability study.

TABLE 6
Related Substances (RS) of Tamsulosin

| | | 1 M, 40° C./ | 2 M, 40° C./ |
|---|---|---|---|
| RS type | Initial | 75% RH | 75% RH |
| R-amine | 0 | 0 | 0 |
| Methoxy analogue | 0 | 0 | 0 |
| Bromide | 0 | 0 | 0 |
| Des Sulphonamide | 0 | 0 | 0 |
| Highest Unknown | 0 | 0.039 | 0.094 |
| Total Unknown | 0 | 0.079 | 0.254 |
| Total RS | 0 | 0.079 | 0.254 |

TABLE 7
Related Substances (RS) of Dutasteride

| | | 1 M, 40° C./ | 2 M, 40° C./ |
|---|---|---|---|
| RS type | Initial | 75% RH | 75% RH |
| Acid Impurity | 0 | 0 | 0 |
| Ester Impurity | 0 | 0 | 0 |
| Beta isomer Impurity | 0.017 | 0.017 | 0.032 |
| Dihydro dutasteride impurity | 0.041 | 0.068 | 0 |
| Highest unknown | 0.013 | 0.029 | 0.021 |
| Total Unknown | 0.031 | 0.089 | 0.042 |
| Total RS | 0.089 | 0.174 | 0.074 |

Results of the stability study indicate that tamsulosin compositions in accordance with the present invention exhibits excellent storage stability.

1. A pharmaceutical composition comprising:
(a) at least one core comprising tamsulosin, or salts thereof;
(b) at least one enteric coating layer coated over the core comprising one or more pharmaceutically acceptable acid resistant polymers,
wherein the mass of said enteric coating layer, calculated on the uncoated core basis is more than 15%.

2. The pharmaceutical composition of claim 1, wherein at least one moisture barrier coating layer is disposed over the enteric coating layer.

3. A fixed dose combination composition comprising:
a first composition, which comprises of—
(a) at least one core comprising tamsulosin, or salts thereof;
(b) at least one enteric coating layer coated over the core comprising one or more pharmaceutically acceptable acid resistant polymers; and
(c) at least one moisture barrier coating layer disposed over the enteric coating layer comprising one or more pharmaceutical polymers, and
a second composition which comprises of dutasteride or salts, hydrates thereof;
characterized in that the mass of said enteric coating layer, calculated on the uncoated core basis is more than 15% and the second composition is physically separated from the first composition.

4. The pharmaceutical composition of claim 1, wherein the acid-resistant acrylic polymer is methacrylic acid and methyl methacrylate copolymer.

5. The pharmaceutical composition of claim 1, wherein the enteric coating layer comprises 20 to 85 mass % of said acid resistant acrylic polymer calculated on the dry layer basis.

6. The pharmaceutical composition of claim 2, wherein the mass of the moisture barrier layer is within the range of about 1 to about 25% w/w of the composition.

7. The pharmaceutical composition of claim, wherein mass of all the coating layers is within the range of about 15 to about 25% w/w calculated on the dry uncoated core basis.

8. The pharmaceutical composition of claim 1, wherein the composition further comprises about 0.05 to about 5.0% w/w of dutasteride or salt thereof.

9. The pharmaceutical composition of claim 1, wherein the composition is in the form of a capsule

10. The pharmaceutical composition of claim 8 prepared by a process, which process comprises steps of:
(a) providing at least one core; comprising tamsulosin or salts thereof;
(b) providing at least one enteric coating layer coated over the core;
(c) providing at least one coating layer disposed over the enteric coating layer providing moisture barrier layer;
(d) providing a second composition comprising dutasteride or salts thereof; and
(e) formulating the first and second compositions in the unit dosage form.

11. A pharmaceutical composition comprising plurality of pellets, which pellets comprises of about 0.05 to about 5.0% w/w of tamsulosin hydrochloride, about 50 to about 85% w/w of microcrystalline cellulose, about 10 to about 25% w/w of the acrylic polymer, about 5 to about 20% w/w polyvinyl alcohol based polymer, and about 0% to about 25% w/w of other pharmaceutical excipients, calculated on the dry pellet basis.

12. The pharmaceutical composition of claim 11, wherein the composition further comprises about 0.05 to about 5.0% w/w of dutasteride or salt thereof.
